(19) United States
(12) Patent Application Publication
(10) Pub. No.: US 2010/0015072 A1
(43) Pub. Date: Jan. 21, 2010

(54) Topical Compositions Comprising Isonicotinamide

(76) Inventors: Christopher Polla, Massapequa, NY (US); John Salto, Deer Park, NY (US); Daniel Korman, East Williston, NY (US); Ashraf Shokralla, New Hyde Park, NY (US); Alaine Ranieri-Chamberlain, Hollsville, NY (US); Ka Wah Josephine Lee, New York, NY (US); Lauren Ingoglia, Huntington, NY (US); James Tobin, Hicksville, NY (US); Edward Pelle, Valley Stream, NY (US)

Correspondence Address: THE ESTEE LAUDER COS, INC, 155 PINELAWN ROAD, STE 345 S, MELVILLE, NY, 11747, US

(21) Appl. No.: 12/486,046

(22) Filed: Jun. 17, 2009

Related U.S. Application Data
(60) Provisional application No. 61/076,800, filed on Jun. 30, 2008.

(51) Int. Cl. A61K 8/49; A61K 31/4409; A61K 36/00

(52) U.S. Cl. 424/60; 514/354; 424/59; 424/725

(57) ABSTRACT

Topical compositions containing isonicotinamide in a cosmetically acceptable carrier and methods for treating keratinous surfaces using the composition.

### CROSS-REFERENCE TO RELATED APPLICATIONS

This application claims priority of U.S. provisional application 61/076,800, filed Jun. 30, 2008.

### TECHNICAL FIELD

The invention is in the field of topical compositions for application to keratinous surfaces for treatment, beautification, or coloring the surface.

### BACKGROUND OF THE INVENTION

Cosmetics companies are always searching for ingredients to include in skin care products that will provide beneficial effects on skin. For example, compounds that activate sirtuins are very desirable for use in cosmetics because these molecules are believed to effect cell health and longevity. Sirtuins are a class of enzymes found in humans and many other organisms. It has been found that they are part of a feedback system that helps to promote cellular survival during times of stress. For example, in feeding studies conducted with worms and mice, it was shown that if the organisms were fed a nutritious diet that contained at least 30% fewer calories than usually consumed, the life span of the organism could be extended by as much as fifty percent. Laboratory studies showed that genetically modified organisms missing the genes that coded for sirtuins did not exhibit the same life span increase with similar caloric restriction. Thus sirtuins appear to be important in stimulating cells to repair damage and prolong function.

There are a number of ingredients that are believed to increase sirtuin activity including resveratrol, a natural component of certain types of grapes. It has also been reported that certain other derivatives of nicotinamide, particularly riboside derivatives of nicotinamide have activity in increasing sirtuins. For example, U.S. Patent Application No. 2007/0117765A1 and 2006/0229265, both incorporated by reference herein in the entirety, are directed to certain nicotinamide riboside derivatives that increase cellular NAD+ levels, which in turn is believed to promote sirtuin levels and otherwise improve cellular health and longevity. These compounds have the ability to improve cellular health and longevity and are very desirable for use in cosmetic compositions, particularly those for treating keratinous surfaces such as skin for improvement.

However, if such sirtuin activators are desired for use in topical compositions applied to skin, they must be formulated into aesthetically pleasing cosmetic compositions. Cosmetics consumers, in particular, are very sophisticated when it comes to tactile and aesthetic properties of the skin care products they purchase. No matter how beneficial to skin a particular ingredient might be, if it can only be delivered to skin in the greasy, thick ointments typically used for over-the-counter pharmaceuticals, it will not be popular with skin care products consumers. Such consumers insist on products that are efficacious as well as aesthetically and tactilely pleasing, especially when these products are expensive. Yet, formulation of products containing active ingredients that have an impact on cellular longevity has many difficulties, one of which is to ensure that the active ingredient in the composition is capable of delivery into the skin. For if the active ingredient that provides these beneficial effects is not stable in the formula, or is not able to penetrate the skin, then it might as well not be present at all.

It is an object of the invention to formulate aesthetically pleasing cosmetic compositions for non-prescription sale to consumers containing ingredients that, when topically applied, promote cellular health and longevity.

It is a further object of the invention to provide commercially acceptable topical cosmetic or pharmaceutical compositions containing isonicotinamide.

It is a further object of the invention to provide commercially acceptable aqueous based or anhydrous topical compositions containing isonicotinamide.

It is a further object of the invention to provide emulsion topical compositions containing isonicotinamide.

It is a further object of the invention to provide commercially acceptable topical compositions for treating skin for improvement comprising at least one isonicotinamide.

It is a further object of the invention to provide topical cosmetic or pharmaceutical compositions in the form of creams, lotions, serums, gels, foundation makeups, and the like, containing at least one isonicotinamide which may be aqueous based or anhydrous.

### SUMMARY OF THE INVENTION

The invention is present in a number of embodiments.

The invention is further directed to aqueous based topical pharmaceutical compositions comprising isonicotinamide and an aqueous phase structuring agent.

The invention is directed to topical compositions comprising isonicotinamide and at least one chemical sunscreen.

The invention is further directed to a topical composition comprising isonicotinamide and at least one antioxidant botanical extract.

The invention is further directed to a topical composition comprising isonicotinamide and at least one silicone elastomer.

The invention is also directed to an emulsion composition comprising water, oil, and isonicotinamide.

The invention is further directed to a method for treating keratinous surfaces such as skin for improvement comprising applying to the surface an aqueous based topical composition containing isonicotinamide.

### DETAILED DESCRIPTION

The compositions of the invention may be in the anhydrous form, or in an aqueous solution or suspension form, or in the water-in-oil or oil-in-water emulsion form. The term “anhydrous” means that water is not intentionally added to the compositions, but it may be present in trace amounts in other ingredients added to the composition. In the case where the composition is aqueous based, the amount of water may range from about 0.1-99%, preferably from about 5-85%, more preferably from about 7-75% by weight of the total composition. In the case where the compositions are emulsions, the amount of oil will preferably range from about 1-95%, preferably from about 5-85%, more preferably from about 7-65% by weight of the total composition.

### I. Isonicotinamide

The term “isonicotinamide” means 4-pyridine carboxamide having the formula:

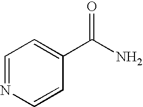

a molecular weight of about 122.2 grams/mol

The compositions of the invention comprise from about 0.001 to 85%, preferably from about 0.01 to 80%, more preferably from about 0.1 to 70% by weight of the total composition.

### II. Aqueous Phase Structuring Agent

In one embodiment, the composition comprising isonicotinamide and at least one aqueous phase structuring agent. The compositions may be in the form of aqueous solutions, dispersions or emulsions. The aqueous phase structuring agent increases the viscosity of the composition. The aqueous phase structuring agent must be compatible with isonicotinamide and also compatible with the other ingredients in the formulation. Suitable ranges of aqueous phase structuring agent, if present, are from about 0.01 to 30%, preferably from about 0.1 to 20%, more preferably from about 0.5 to 15% by weight of the total composition. Examples of such agents include various acrylate based thickening agents, natural or synthetic gums, polysaccharides, and the like, including but not limited to those set forth below. Isonicotinamide is generally water soluble, and, in conjunction with the aqueous phase thickening agent, also contributes to stabilizing this ingredient in the composition and improving penetration into the stratum corneum.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylate based polymeric thickeners comprised monomers such as acrylic acid, methacrylic acid, or their simple C₁₋₂₂ carboxylic acid esters; acrylamides; ammonium acrylate or methacrylate; dimethylaminoethylmethacrylate; PPG-diacrylate; styrene; Examples include acrylates copolymer sold under the trademark Capigel; Polyacrylate 1, 2, 3, 4, 5, 6, 7, 8, 9, 10, 11, 12, 13, 14, 15, 16, 17, 18, 19, 20, 21, 22; polyacrylate-1 crosspolymer; polyacrylate-2 crosspolymer; steareth-10 allyl ether/acrylates copolymer; carbomer; C10-30 alkyl acrylates crosspolymer or carbomer; or acrylate based polymer thickeners sold by Clariant under the Aristoflex trademark such as ammonium acryloyldimethyltaurate/VP copolymer; or ammonium acryloyldimethyltaurate/beheneth-25 methacrylate crosspolymer; and the like.

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

### III. Oils

In the event the compositions of the invention are in emulsion form, the composition will comprise an oil phase. Oily ingredients are desirable for the skin moisturizing and protective properties. Oils, if present, may assist in forming a barrier on the skin so that the isonicotinamide present in the composition remains on the skin. Suitable oils include silicones, esters, vegetable oils, synthetic oils, including but not limited to those set forth herein. The oils may be volatile or nonvolatile, and are preferably in the form of a pourable liquid at room temperature. The term “volatile” means that the oil has a measurable vapor pressure or a vapor pressure of at least about 2 mm. of mercury at 20° C. The term “nonvolatile” means that the oil has a vapor pressure of less than about 2 mm. of mercury at 20° C.

Suitable volatile oils generally have a viscosity ranging from about 0.5 to 5 centistokes 25° C. and include linear silicones, cyclic silicones, paraffinic hydrocarbons, or mixtures thereof. Volatile oils may be used to promote more rapid drying of the skin care composition after it is applied to skin. Volatile oils are more desirable when the skin care products containing the isonicotinamide are being formulated for consumers that have combination or oily skin. The term “combination” with respect to skin type means skin that is oily in some places on the face (such as the T-zone) and normal in others.

Cyclic or branched volatile silicones may be used in the composition. Such silicones have the general formula:

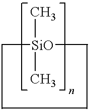

where n=3-6, preferably 4, 5, or 6.

Also suitable are linear volatile silicones, for example, those having the general formula:

(CH₃)₃Si—O—[Si(CH₃)₂O]—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the trade names Dow Corning 244, 245, 344, and 200 fluids. These fluids include hexamethyldisilazane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

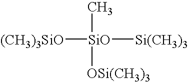

Methyl trimethicone may be purchased from Shin-Etsu Silicones under the tradename TMF-1.5, having a viscosity of 1.5 centistokes at 25° C.

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference.

Preferred volatile paraffinic hydrocarbons have a molecular weight of 70-225, and may be available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Suitable esters are mono-, di-, and triesters. The composition may comprise one or more esters selected from the group, or mixtures thereof.

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyidodecyl citrate, tridecyl behenate; or tridecyl cocoate, tridecyl isononanoate, and so on.

Nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof.

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

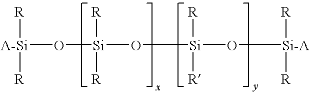

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

### IV. Oil Phase Structuring Agents

In the case where the composition is anhydrous or in the form of an emulsion, it may be desirable to include one or more oil phase structuring agents in the cosmetic composition. The term “oil phase structuring agent” means an ingredient or combination of ingredients, soluble or dispersible in the oil phase, which will increase the viscosity, or structure, the oil phase. The oil phase structuring agent is compatible with the isonicotinamide, particularly if the isonicotinamide is soluble in the nonpolar oils forming the oil phase of the composition. The term “compatible” means that the oil phase structuring agent and isonicotinamide derivative are capable of being formulated into a cosmetic product that is generally stable. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suggested ranges of structuring agent are from about 0.01 to 70%, preferably from about 0.05 to 50%, more preferably from about 0.1-35% by weight of the total composition. Suitable oil phase structuring agents include those that are silicone based or organic based. They may be polymers or non-polymers, synthetic, natural, or a combination of both.

A. Silicone Structuring Agents

A variety of oil phase structuring agents may be silicone based, such as silicone elastomers, silicone gums, silicone waxes, linear silicones having a degree of polymerization that provides the silicone with a degree of viscosity such that when incorporated into the cosmetic composition it is capable of increasing the viscosity of the oil phase. Examples of silicone structuring agents include, but are not limited to:

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the CTFA name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety. It is particularly desirable to incorporate silicone elastomers into the compositions of the invention because they provide excellent “feel” to the composition, are very stable in cosmetic formulations, and relatively inexpensive.

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

The silicone gums that are used in the compositions include, but are not limited to, those of the general formula wherein:

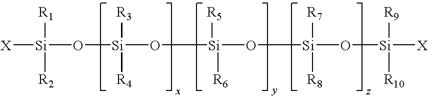

R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the CTFA name dimethicone.

Another type of oily phase structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

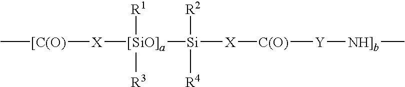

X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

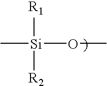

### and Y is:

(a) a linear or branched alkylene having from about 1-40 carbon atoms which may be substituted with:
(i) one or more amide groups having the general formula R₁CONR₁, or
(ii) C₅₋₆ cyclic ring, or
(iii) phenylene which may be substituted with one or more C₁₋₁₀-alkyl groups, or
(iv) hydroxy, or
(v) C₃₋₈ cycloalkane, or
(vi) C₁₋₂₀ alkyl which may be substituted with one or more hydroxy groups, or
(vii) C₁₋₁₀ alkyl amines; or

(b) TR₅R₆R₇
wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

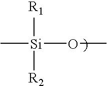

Preferred is where R₁, R₂, R₃, and R₄ are C₁₋₁₀, preferably methyl; and X and Y is a linear or branched alkylene. Preferred are silicone polyamides having the general formula

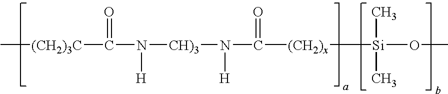

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

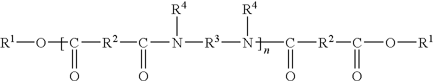

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C30-42 hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCl name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

Also suitable as the oil phase structuring agent may be one or more natural or synthetic waxes such as animal, vegetable, or mineral waxes. Preferably such waxes will have a higher melting point such as from about 50 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

### V. Surfactants

The composition may contain one or more surfactants, especially if in the emulsion form. Such surfactants may be silicone or organic based. The surfactants will aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

Suitable silicone surfactants include polyorganosiloxane polymers that have amphiphilic properties, for example contain hydrophilic radicals and lipophilic radicals. These silicone surfactants may be liquids or solids at room temperature.

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

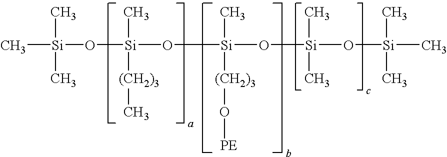

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

### VI. Humectants

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol.

### VII. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, Padina Pavonica extract, thermus thermophilis ferment extract, camelina sativa seed oil, boswellia serrata extract, olive extract, Aribodopsis Thaliana extract, Acacia Dealbata extract, Acer Saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, Panax Ginseng, Siegesbeckia Orientalis, Fructus Mume, Ascophyllum Nodosum, Bifida Ferment lysate, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, and mixtures thereof.

### VIII. Sunscreens

It may also be desirable to include one or more sunscreens in the compositions of the invention. Such sunscreens include chemical UVA or UVB sunscreens or physical sunscreens in the particulate form. Inclusion of sunscreens in the compositions containing the isonicotinamide will provide additional protection to skin during daylight hours and promote the effectiveness of the isonicotinamide on the skin.

A. UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula:

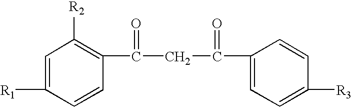

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

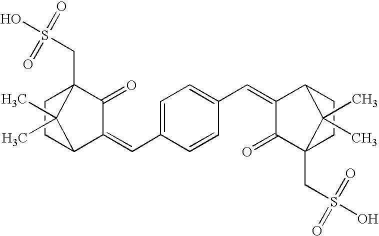

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

B. UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

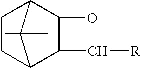

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

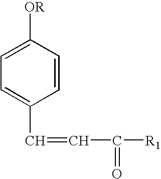

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

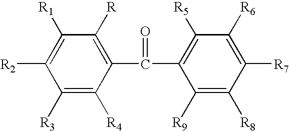

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

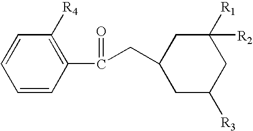

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

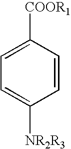

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have a certain SPF (sun protective factor) values ranging from about 1-50, preferably about 2-45, most preferably about 5-30. Calculation of SPF values is well known in the art.

### IX. Particulate Materials

The compositions of the invention may contain particulate materials in the form of pigments, inert particulates, or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

A. Powders

The particulate matter may be colored or non-colored (for example white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

B. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

### X. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

### XI. Vitamins and Antioxidants

The compositions of the invention may contain vitamins and/or coenzymes, as well as antioxidants. If so, 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and so on; the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are retinyl palmitate, retinol. retinoic acid, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### XII. The Cosmetic Compositions

The compositions of the invention containing the isonicotinamides may be found in a variety of forms, such as anhydrous compositions, aqueous based solutions, serums, gels, skin creams or lotions, or color cosmetic compositions such as foundation makeup, mascara, lip color, blush, eyeshadow, and the like. In the case where the composition is in the anhydrous form the isonicotinamide may be solubilized or dispersed in the oil phase of the emulsion; or if the isonicotinamide is water soluble it may be solvated in polar solvents, typically ingredients referred to as humectants such as glycerine or alkylene glycols prior to formation of an anhydrous emulsion.

If the composition is in the emulsion form, the isonicotinamide may be found in the water phase or the oil phase of the emulsion depending on the type of derivative. For example, certain hydrophilic derivatives such as isonicotinamide acetate salt and the like are water soluble and will generally be solubilized in the water phase of the emulsion. Certain other derivatives are lipophilic in nature and will more likely be found in the oil phase of the emulsion.

Suitable serums or gels will generally comprise from about 1-99% water, and optionally from about 0.001-30% of an aqueous phase thickening agent. The other ingredients mentioned herein may be present in the percentage ranges set forth.

Typical skin creams or lotions comprise from about 5-98% water, 1-85% oil, and from about 0.1 to 20% of one or more surfactants. Preferably the surfactants are nonionic and may be in the form of silicones or organic nonionic surfactants.

Typical color cosmetic compositions such as foundations, blush, eyeshadow and the like will preferably contain from about 5-98% water, 1-85% oil, and from about 0.1 to 20% of one or more surfactants in addition to from about 0.1 to 65% of particulates that are pigments or a combination of pigments and powders.

Typical mascara compositions generally contain from about 5-98% water, 1-85% oil, and from about 0.1 to 20% surfactant in addition to natural or synthetic polymers that are film forming, such as aqueous dispersions of acrylic copolymers, aqueous dispersions of polyurethane, or silicone resins.

### XIII. The Methods

The invention further comprises treating skin for improvement by applying to the skin the compositions of the invention. The compositions may be applied in the forms mentioned herein, as part of skin care regimens. For example, the composition may be applied to the skin as a night cream or cream applied to skin prior to a period of bodily rest such as a nap or sleep. The composition may be applied two times a day, in the morning and in the evening after cleansing the skin. The composition may be applied to the skin over skin care products, in the form of foundations or other color cosmetics.

In one embodiment, the isonicotinamide is formulated into a day cream and a night cream, so that the consumer using the regimen applies the isonicotinamide to the skin twice a day as part of a standard skin care routine.

In another embodiment, the isonicotinamide is applied to the skin in the form of a toner, over which a skin cream or lotion is applied.

In another embodiment the isonicotinamide is applied to the skin in the form of a skin cleanser.

The term “treating skin for improvement” means that the skin to which the composition is applied will exhibit one or more of improvements such as increase in skin tone (reduction of laxity), improvement in hyperpigmentation such as age spots, sun spots, or other skin pigmentation associated with aging skin, improvement in the appearance of lines and/or wrinkles, improvement in skin mottling, cellular regeneration, keratolysis (e.g. sloughing off or renewal of keratinocytes),

The invention will be further described in connection with the following examples which are set forth for the purposes of illustration only.

### Example 1

Compositions according to the invention were made as follows:

| | % by weight | | | |
|---|---|---|---|---|
| Ingredient | 1 | 2 | 3 | 4 |
| Water | QS | QS | QS | QS |
| Pentaerythrityl | 5.00 | | | 1.40 |
| tetraoctanoate | | | | |
| Ethylhexylmethoxycinnamate | | 7.50 | 7.50 | 7.50 |
| Ethylhexyl salicylate | | | | 3.50 |
| Cetyl ethylhexanoate | | 3.50 | 3.50 | |
| Neopentylglycol diheptanoate | | | 3.60 | |
| Dimethicone/polysilicone-11 | | 8.00 | | 1.00 |
| Squalane | | 5.50 | | |
| Potato starch | | | | 1.00 |
| Octyl palmitate | | 4.00 | | |
| PEG-8 | | 4.00 | | |
| Di C12-15 alkyl fumarate | | 3.00 | | |
| PEG-100 stearate/glyceryl stearate | | 2.60 | 5.00 | 0.85 |
| Methyl gluceth-20 | | 2.00 | | |
| Cyclopenta- | 5.00 | | | |
| siloxane/ | | | | |
| dimethicone | | | | |
| Cetyl alcohol | | 1.60 | 0.75 | |
| Behenyl alcohol | | | | 0.625 |
| Tocopheryl acetate | | 0.50 | | 0.50 |
| Hexadecyl | 4.00 | | | |
| stearate | | | | |
| Tromethamine | | 0.28 | | |
| Hydrogenated lecithin | | 1.00 | 1.00 | 1.50 |
| Hydrogenated lecithin/C12-16 | | | | 0.50 |
| alcohols/palmitic acid | | | | |
| Caprylyl | | 0.70 | 1.00 | 0.70 |
| glycol/phenoxyethanol/hexylene | | | | |
| glycol | | | | |
| Polyethylene | | | 1.00 | 1.00 |
| Glycerin | 2.50 | | 1.00 | 2.00 |
| Citric acid | | | | 0.06 |
| Butylene | 2.50 | 7.10 | 2.00 | 1.50 |
| glycol | | | | |
| Caffeine | | | | 0.20 |
| Jojoba seed oil | 2.50 | | | 2.00 |
| Aloe Barbadensis leaf extract | | 1.00 | | |
| Glyceryl stearate | 1.90 | 0.40 | 1.25 | 0.50 |
| Cetearyl alcohol/ | 1.50 | | | |
| cetearyl | | | | |
| glucoside | | | | |
| Octyldodecyl neopentanoate | | | | 1.305 |
| Hydrogenated | 1.25 | | | |
| lecithin | | | | |
| Acrylamide/ | 1.15 | | | |
| Sodium acryloylde- | | | | |
| methyltaurate | | | | |
| copolymer/ | | | | |
| isohexadecane/ | | | | |
| polysorbate 80 | | | | |
| Ammonium | | | | 0.70 |
| acryloyldimethyltaurate/VP | | | | |
| copolymer | | | | |
| Carbomer | | 0.37 | 0.36 | |
| Potassium hydroxide | | | 0.24 | |
| Cetyl ricinoleate | 1.00 | | | |
| PEG-100 | 1.00 | 0.30 | 1.25 | |
| stearate | | | | |
| Algae extract | | | 0.001 | 0.001 |
| Polygonum cuspidatum root | 1.00 | | 1.00 | 1.00 |
| extract/ | | | | |
| declustered water/saccharomyces | | | | |
| lysate extract | | | | |
| Saccharomyces lysate | 1.00 | | | |
| extract/declustered water/Camellia | | | | |
| Sinensis (yellow tea) extract/ | | | | |
| Camellia Sinensis (white tea) leaf | | | | |
| extract, Asplanathus | | | | |
| Water/glycine/ | 1.00 | | 0.10 | 1.00 |
| carbomer/hydroxyl- | | | | |
| proine/proline/ | | | | |
| acrylates copolymer | | | | |
| Petrolatum | 1.00 | | | 2.00 |
| DiC12-15 alkyl fumarate | | | | 1.80 |
| Sorbitol | 1.00 | | 0.10 | 1.00 |
| Butylene glycol | | | 2.00 | 1.50 |
| Dimethicone | 1.00 | 2.00 | | 0.80 |
| Polyceryl-3 | 0.75 | | | |
| beeswax | | | | |
| Caprylyl glycol/ | 0.70 | | 1.00 | |
| phenoxyethanol/ | | | | |
| hexylene glycol | | | | |
| Phenoxyethanol | 0.50 | | | 0.013 |
| Isonicotinamide | 0.50 | 0.50 | 0.50 | 0.50 |
| Pentylene glycol | | | 1.00 | 1.50 |
| Behenyl alcohol | | | 0.95 | |
| Lecithin | | | 0.20 | |
| Hydrogenated polyisobutene | | | | 1.40 |
| Hexadecyl stearate | | | | 1.40 |
| Water/hydrogenated | 0.50 | | | |
| lecithin/ascorbyl tocopheryl | | | | |
| maleate/rosmarinus | | | | |
| officinalis/nordihydroguaiaretic | | | | |
| acid/linoleic acid/linolenic acid | | | | |
| Water/Artemia extract | 0.50 | | | |
| Glycine soya (soybean) | 0.50 | | | |
| extract | | | | |
| Water/butylene glycol/disodium | 0.50 | | 1.00 | 0.50 |
| NADH/lecithin/lauryldimonium | | | | |
| hydroxypropyl hydrolyzed soy | | | | |
| protein/caprylyl glycol/xanthan | | | | |
| gum/adenosine | | | | |
| phosphate/creatine/ | | | | |
| phenoxyethanol/hexylene | | | | |
| glycol | | | | |
| Betaine | 0.50 | | | |
| Tocopheryl acetate | | 0.50 | | |
| Trehalose | 0.50 | | 0.10 | 0.10 |
| Water/saccharomyces lysate | 0.50 | | | |
| extract/Centella Asiatica extract | | | | |
| Wheat (Triticum Vulgare) Bran | | | 0.20 | |
| extract/Olive (Olea) Europaea | | | | |
| extract | | | | |
| Aminoguanidine HCL | | | 0.20 | 0.20 |
| Hydroxyapatite | 0.50 | | | |
| Butylene glycol/water/ | 0.50 | | | |
| Matricaria extract | | | | |
| Sucrose | 0.50 | 0.50 | | |
| Myristyl myristate | 0.50 | | | |
| Tocopheryl acetate | 0.50 | | | |
| Water/sodium hydroxide | | | | 0.085 |
| Butylene glycol/Scutellaria | 0.30 | | | |
| Baicalensis Root extract/Morus | | | | |
| Bombycis Root extract | | | | |
| Water/Acetyl hexapeptide-8 | 0.25 | | 0.25 | 0.25 |
| Acrylates/C10-30 alkyl acrylate | 0.24 | | | |
| crosspolymer | | | | |
| Xanthan gum | | | 0.15 | 0.15 |
| Caffeine | 0.20 | 0.20 | 0.20 | |
| Linoleic acid | 0.20 | 0.10 | 0.20 | 0.20 |
| Titanium dioxide/aluminum | | 3.00 | 3.00 | |
| hydroxide/stearic acid/octyldodecyl | | | | |
| neopentanoate/glyceryl stearate | | | | |
| Titanium dioxide/methicone | | | | 1.305 |
| Water/butylene glycol/citrus | | 0.10 | | |
| jabara peel extract | | | | |
| Water/butylene glycol/hydrolyzed | 0.10 | 0.05 | 0.05 | |
| rice extract | | | | |
| Aminoguanidine HCL | 0.20 | | 0.20 | |
| Phytosphingosine/propylene glycol | 0.20 | | | |
| dicaprate | | | | |
| Squalane/Hordeum Vulgare | 0.20 | | | 0.20 |
| (Barley) extract/Triticum Vulgare | | | | |
| (wheat) germ extract | | | | |
| Cholesterol | 0.20 | | 0.20 | 0.20 |
| Cholesterol/potassium sulfate | | | 0.10 | 0.05 |
| Tromethane | 0.20 | | | |
| Disodium EDTA | 0.10 | 0.10 | 0.20 | 0.10 |
| Sodium Ribonucleic acid | 0.10 | | 0.10 | 0.10 |
| Potassium sorbate | 0.10 | | | 0.10 |
| Glycerin/Padina Pavonica extract | 0.10 | | 0.10 | 0.10 |
| Water/decarboxy carnosine | 0.10 | | 0.10 | 0.10 |
| HCL/butylene glycol | | | | |
| Siegesbeckia Orientalis | 0.10 | | 0.10 | 0.10 |
| extract/glycerin | | | | |
| Dipalmitoyl hydroxyproline | 0.10 | | 0.10 | 0.10 |
| Water/Ethylhexyl | 0.10 | | 0.10 | 0.10 |
| palmitate/butylene | | | | |
| glycol/cholesterol/Astrocaryum | | | | |
| Murmuru butter/linoleic acid/ | | | | |
| coffea robusta seed | | | | |
| extract/ethylhexyl | | | | |
| stearate/carbomer/propylene glycol | | | | |
| dicaprate/phytosphingosine | | | | |
| Propylene glycol | | 0.20 | | |
| dicaprate/Helianthus Annus | | | | |
| (sunflower) seed cake/Hordeum | | | | |
| Vulgare (Barley) extract/Cucumis | | | | |
| Sativus (Cucumber) fruit extract | | | | |
| Inonotus Obliquus (mushroom) | 0.10 | | | |
| extract/cellulose | | | | |
| Phenoxyethanol | 0.08 | 0.01 | 0.135 | |
| Cholesterol/potassium sulfate | 0.05 | | | |
| Declustered water/Betula Alba | 0.05 | | 0.05 | |
| (birch) bark extract/saccharomyces | | | | |
| lysate extract | | | | |
| Coleus Barbatus extract | 0.05 | | 0.05 | 0.05 |
| Water/butylene glycol/hydrolyzed | 0.05 | | 0.05 | 0.05 |
| rice extract | | | | |
| Aminopropyl ascorbyl phosphate | 0.045 | | 0.035 | 0.045 |
| Sodium hyaluronate | 0.03 | 0.005 | 0.05 | 0.015 |
| Malt extract | 0.0147 | | | |
| Adenosine phosphate | 0.010 | | | |
| Citri Reticulatae peel extract | 0.0001 | | 0.0001 | 0.0001 |
| Simethicone | 0.000097 | | | |
| Silica | | | 1.00 | |

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

What we claimed is:

1. An aqueous based topical composition comprising isonicotinamide and an aqueous phase structuring agent which is an acrylate based synthetic polymer.

2. The composition of claim 1 wherein the aqueous phase structuring agent comprises an acrylate based synthetic polymer selected from the group consisting of.

3. The composition of claim 2 wherein the acrylate based synthetic polymer comprises monomers selected from acrylic acid, methacrylic acid, their simple C₁₋₂₂ carboxylic acid esters; acrylamides; ammonium acrylate or methacrylate; dimethylaminoethylmethacrylate; PPG-diacrylate; styrene; or mixtures thereof.

4. The composition of claim 2 wherein the synthetic polymer is selected from the group consisting of Acrylates Copolymer; Polyacrylate 1; Polyacrylate 2; Polyacrylate 3; Polyacrylate 4; Polyacrylate 5; Polyacrylate 6; Polyacrylate 7; Polyacrylate 8; Polyacrylate 9; Polyacrylate 10; Polyacrylate 11; Polyacrylate 12; Polyacrylate 13; Polyacrylate 14; Polyacrylate 15; Polyacrylate 16; Polyacrylate 17; Polyacrylate 18; Polyacrylate 19; Polyacrylate 20; Polyacrylate 21; Polyacrylate 22; Polyacrylate-1 Crosspolymer; Polyacrylate-2 Crosspolymer; Steareth-10 Allyl Ether/Acrylates Copolymer; Carbomer; C10-30 Alkyl Acrylates Crosspolymer; Ammonium Acryloyldimethyltaurate/VP Copolymer; or Ammonium Acryloyldimethyltaurate/Beheneth-25 Methacrylate Crosspolymer; and mixtures thereof.

5. The composition of claim 4 which is an oil in water emulsion comprising isonicotinamide; an acrylate based synthetic polymeric aqueous phase thickening agent; at least one monoester; at least one botanical extract; at least one volatile silicone; at least one nonovolatile silicone; and at least one humectant which is glycerin or an alkylene glycol.

6. An aqueous based topical composition comprising isonicotinamide and at least one chemical sunscreen selected from UVA sunscreens, UVB sunscreens, or mixtures thereof

7. The composition of claim 6 further comprising at least one silicone elastomer; at least one nonvolatile silicone; at least one acrylate based synthetic polymer aqueous phase structuring agent; at least one glyceryl ester of a fatty acid; at least one humectant which is glycerin, an alkylene glycol, or mixtures thereof, at least one botanical extract.

8. The composition of claim 7 wherein the silicone elastomer comprises Polysilicone-11; the nonvolatile silicone comprises dimethicone; the acrylate based structuring agent comprises carbomer; the glyceryl ester of fatty acid comprises glyceryl stearate; the humectant comprises glycerin; and the botanical extract comprises one or more of Aloe Barbadensis leaf extract, Polygonum Cuspidatum extract, Camellia Sinensis; Alplanathus Linearis; Rosmarinus Officinalis; Soybean; Artemia; Centella Asiatica; Bran extract; Olive extract; Matricaria extract; Scutellaria Baicalensis extract; Morus Bombycis extract; Citrus Jabara Peel Extract; Barley extract; Wheat germ; Padina Pavonica; Siegesbeckia Orientalis; Coffea Robusta; Helianthus Annus; Cucumis Sativus; Inonotus Obliquus; Betula Alba; Coleus Barbatus; Citri Reticulatae; and mixtures thereof.

9. The composition of claim 8 wherein the UVB sunscreen comprises ethylhexylmethoxycinnamate, ethylhexylsalicylate, or mixtures thereof.

10. The composition of claim 8 in the form of an oil in water emulsion.

11. A topical composition comprising isonicotinamide and at least one botanical extract.

12. The composition of claim 11 wherein the antioxidant botanic extract is selected from the group consisting of Aloe Barbadensis leaf extract, Polygonum Cuspidatum extract, Camellia Sinensis; Alplanathus Linearis; Rosmarinus Officinalis; Soybean; Artemia; Centella Asiatica; Bran extract; Olive extract; Matricaria extract; Scutellaria Baicalensis extract; Morus Bombycis extract; Citrus Jabara Peel Extract; Barley extract; Wheat germ; Padina Pavonica; Siegesbeckia Orientalis; Coffea Robusta; Helianthus Annus; Cucumis Sativus; Inonotus Obliquus; Betula Alba; Coleus Barbatus; Citri Reticulatae; and mixtures thereof.

13. The composition of claim 12 additionally comprising at least one chemical sunscreen; at least one ester; at least one glyceryl ester of a fatty acid; at least one humectant selected from an alkylene glycol or glycerin; and at least one acrylate based synthetic polymeric thickening agent.

14. The composition of claim 13 wherein the glyceryl ester is glyceryl stearate, PEG-100 stearate or mixtures thereof, the humectant comprises glycerin; and the acrylate based synthetic polymer comprises carbomer.

15. The composition of claim 12 further comprising at least one silicone elastomer.

16. The composition of claim 12 further comprising glycerin.

17. The composition of claim 12 further comprising dimethicone.

18. The composition of claim 12 further comprising at least one acrylate based synthetic polymer structuring agent.

19. The composition of claim 18 wherein the structuring agent comprises acrylates C10-30 alky acrylates crosspolymer.

20. The composition of claim 18 wherein the structuring agent comprises carbomer.
